(19) United States
(12) Patent Application Publication
(10) Pub. No.: US 2025/0307277 A1
(43) Pub. Date: Oct. 2, 2025

(54) Real-Time and Diagnostic Omnichannel Interaction Insights, Actions, and Management Using Machine Learning Models

(71) Applicant: Elevance Health, Inc., Indianapolis, IN (US)

(72) Inventors: Vikram Bandugula, Chicago, IL (US); Haoyun Feng, Rye Brook, NY (US); Sanjay Yermalkar, Buffalo Grove, IL (US); Ayush Mathur, Novi, MI (US); Hrushikesh Dhumal, Broadlands, VA (US); Sumant Bhandari, Barrington, IL (US); Umesh Rath, Atlanta, GA (US)

(21) Appl. No.: 18/939,258

(22) Filed: Nov. 6, 2024

Related U.S. Application Data
(60) Provisional application No. 63/572,151, filed on Mar. 29, 2024.

(51) Int. Cl. G06F 16/332; G06F 16/335; G06F 40/295

(52) U.S. Cl. CPC G06F 16/3329; G06F 16/3328; G06F 16/335; G06F 40/295

(57) ABSTRACT

Systems, methods and user interfaces are provided for generating real-time and/or diagnostic omnichannel interaction insights. The method may include obtaining transcripts corresponding to digital service channels. The method may also include generating and inputting channel-specific prompts to machine learning models to obtain insights. The method may also include generating and/or displaying analytical insights. The method may also include obtaining a natural language question, via a conversational interface, directed to a benefits database. The method may also include parsing the question. The method may also include ranking benefits using a recommendation algorithm. The method may also include generating a context by applying a language template. The method may also include inputting the context to a large language model. The method may also include providing a response to an agent to cause the agent to perform one or more actions. The method may also include generating and displaying a dashboard.

### CROSS-REFERENCE TO RELATED APPLICATION

This application claims the benefit of U.S. Provisional Patent Application No. 63/572,151, filed Mar. 29, 2024, entitled “Real-Time and Diagnostic Omnichannel Interaction Insights, Actions, and Management Using Machine Learning Models,” which is incorporated by reference herein in its entirety.

### BACKGROUND

Omnichannel interaction has significant potential benefits for organizations. Omnichannel interaction may include multiple channels, such as multichannel and cross-channel interaction. The channels may be connected, interconnected and/or interactive. The channels may simultaneously exchange information across multiple interaction channels. Omnichannel interactions may also include online and offline interactions. Omnichannel interactions in health care services can provide a seamless and personalized experience for patients. Technologies for omnichannel interaction can help improve patient engagement and satisfaction, healthcare access, and provide cost savings. Omnichannel interactions can lead to actions, insights and management operations that may need to be provided in real-time and/or for diagnostic purposes.

### SUMMARY

Accordingly, there is a need for systems, methods and interfaces that provide real-time and/or diagnostic insights, actions, and management operations for omnichannel interactions. The techniques described herein can enable efficiencies in call centers, for example. For instance, agents may handle stakeholder interactions from calls and/or chats in a more efficient manner with access to real-time (or near real-time) information. The systems according to the techniques described herein may provide contextual insights across interactions. The insights can enable stakeholders understand insights at scale based on contextual outputs. Some embodiments consume a large volume of health plan related content (e.g., structured, unstructured, across numerous data sources). Some embodiments generate output that may be leveraged by agents, healthcare providers and/or members. Contextualization of complex data and/or documentation of health plan may generate meaningful output. Some embodiments provide interaction level insights on complex language/interaction content. In contrast to conventional systems, calls may be automatically reviewed to understand if agents interacted empathetically and/or accurately with an external stakeholder (e.g., member, provider, broker).

In one aspect, a method is provided for generating real-time and diagnostic omnichannel interaction insights. The method may include obtaining one or more transcripts corresponding to a plurality of digital service channels. The method may also include generating one or more channel-specific prompts for the one or more transcripts based on each digital service channel corresponding to a respective transcript and metadata extracted from the one or more transcripts. The method may also include inputting the one or more channel-specific prompts to one or more machine learning models to obtain channel-specific insights. The method may also include generating and displaying analytical insights by integrating the channel-specific insights with member-specific healthcare data, using sentiment analysis and data filtering.

In some embodiments, generating the one or more channel-specific prompts is based on a prompt library for different digital service channels. The prompt library may include domain-specific prompt engineering resources and libraries to devise prompts relevant for domain-specific dialogues.

In some embodiments, generating the one or more channel-specific prompts is based on: analyzing common queries or intents in domain-specific omnichannel interaction data to identify frequently occurring query patterns, intents, and topics that domain users express; using intent classification on domain-specific omnichannel interaction data to categorize utterances into distinct buckets like benefits inquiry, claims assistance and provider search to use intent categories to generate prompts; using named entity recognition (NER) to extract entities like medication names, treatment procedures, and insurance terms, in healthcare omnichannel interaction data to frame prompts incorporating the entities; analyzing and/or reverse engineering prompt-response pairs from prior domain-specific omnichannel interaction data to discern patterns and templates for new prompts; using input from domain experts like physicians, nurses and claims specialists to use domain knowledge to suggest prompts spanning different healthcare scenarios and contexts; and/or performing A/B tests with candidate prompts with a large language model to assess response quality, clarity, specificity and adherence to healthcare compliance, to iteratively refine prompts based on test results.

In some embodiments, applying one or more machine learning models includes performing in-memory analysis of transcript segments of the one or more transcripts.

In some embodiments, the one or more machine learning models are trained on healthcare terminology, medications and/or treatments to output healthcare domain-specific data.

In some embodiments, the plurality of health service channels includes two or more channels selected from the group consisting of: (i) a phone channel for interaction with agents trained to respond about benefits, claims and providers; (ii) an email or secure messaging channel for written inquiries about benefits, claims, healthcare documents, including attachments for evidence of claim, explanation of benefits statements; (iii) a chat or instant messaging channel for real-time interaction to obtain healthcare related information; (iv) a web portal channel for secure online accounts to view benefits, check claim status, order identifier cards, updating contact information, uploading claims and documents; and (v) a social media channel for responding to public inquiries, providing updates during events impacting members.

In some embodiments, the one or more transcripts include: (i) text related to healthcare topics including claims, benefits, insurance plans, coverage, medical terminology, and regulations; (ii) at least some data with protected health information; (iii) communication between patients, insurance companies and healthcare providers; and (iv) speech and text data, including telephonic operations and call recordings.

In some embodiments, obtaining the one or more transcripts includes interfacing with one or more third-party provider computers to receive text and/or speech data.

In some embodiments, the method further includes storing the one or more transcripts in a cloud object storage, and cataloging and storing the metadata in a NoSQL database or persistent key-value datastore for replication, autoscaling, encryption at test, and on-demand backup.

In some embodiments, performing the in-memory analysis includes, upon availability of the metadata, initiating a Kubeflow-based job, deploying a plurality of pods, each pod processing transcript segments, based on metadata from a document database and the one or more transcripts.

In some embodiments, the method further includes storing, by the plurality of pods, the channel-specific insights in a cloud object storage as encrypted files, using 256 AES encryption.

In some embodiments, obtaining the one or more transcripts includes optimizing transcript processing using a read-optimized document database as an intermediary cache, wherein an hourly Extract, Transform and Load (ETL) job, orchestrated via Airflow, activates either a transient Elastic Map Reduce (EMR) cluster or an Amazon Web Services (AWS) Glue job, thereby identifying and migrating new records into a database.

In some embodiments, applying data filtering includes filtering the call-specific insights and the member-specific data by a plurality of parameters, including time, topic, and sentiment.

In some embodiments, the method further includes generating a data visualization based on the analytical insights, the data visualization subject to a predetermined latency.

In some embodiments, the method further includes indexing the analytical insights and/or the one or more transcripts chronologically for real-time querying.

In some embodiments, the method further includes providing one or more application programming interfaces (APIs) for (i) retrieving and/or (ii) interpreting user interface filters to query, the one or more call transcripts, the call-specific insights, and/or the analytical insights.

In some embodiments, the one or more transcripts combines text or speech data obtained from the plurality of health service channels. At least two of the plurality of health service channels may generate text or speech in distinct format or structure.

In some embodiments, the one or more transcripts include text that is protected health information that is anonymized and is compliant with HIPAA and/or privacy regulations.

In some embodiments, generating the analytical insights comprises integrating interactions across multiple health service channels for a same member over time.

In some embodiments, the one or more machine learning models are trained to identify healthcare related topics and/or sub-topics within each transcript.

In some embodiments, the one or more machine learning models are trained to identify member sentiment for each transcript, using sentiment analysis.

In some embodiments, the one or more machine learning models are trained to generate a summary for each transcript or the one or more transcripts.

In some embodiments, the transcript includes labeled speakers at least one of which is a healthcare service agent and another is a member of a healthcare service.

In some embodiments, generating the analytical insights includes generating an issue resolution flag for a transcript to indicate if an issue raised in the transcript has been resolved in an interaction corresponding to the transcript.

In some embodiments, generating the analytical insights includes identifying a unique member identification for healthcare service members.

In another aspect, a method is provided for generating enhanced domain-specific analytics. The method includes obtaining a natural language question, via a conversational interface, directed to a benefits database. The method also includes parsing the natural language question to identify a user intent. The method also includes ranking one or more benefits in the benefits database by inputting the user intent to a recommendation algorithm to obtain structured data. The method also includes generating a context by applying a language template to the structured data. The method also includes inputting the context to a trained large language model to generate a response to the natural language question. The method also includes providing, via the conversational interface, the response to an agent to cause the agent to perform one or more actions. The method also includes generating and displaying a dashboard showing the user intent, the one or more actions, and a sentiment resulting from performing the one or more actions.

In some embodiments, the recommendation algorithm is learning to rank algorithm that is trained by: preparing benefits data related to various benefits, their descriptions, coverage details, eligibility criteria, historical data on how users have searched and interacted with different benefits in the benefits database, by extracting query-benefit pairs along with relevance ratings from user interactions or expert annotations; extracting one or more features from the benefits data that influence ranking, such as benefit type, coverage scope, cost-sharing details, provider network, applicable conditions/treatments, query features like keyword matches, semantic similarity with benefit text, user profile signals, query and benefit attributes; and using the extracted features to train a learning to rank (LTR) model like LambdaRank, RankNet, or ListNet to learn a ranking function that optimizes for a desired metric (e.g., NDCG) by minimizing the loss between predicted and true relevance rankings for benefits.

In some embodiments, generating the response to the natural language question further comprises integrating analytical information based on omnichannel interaction data obtained from a plurality of digital service channels for interaction with a plurality of members.

In some embodiments, the language template includes one or more templates selected from the group consisting of: descriptive sentence templates including benefit names, coverage details, eligible conditions or treatments, plan types; question-answer templates including benefit names, coverage details, eligibility criteria, cost-sharing details; conversational templates including benefit names, plan types, conditions or treatments; structured key-value templates including benefit name, coverage, eligibility, cost, providers, plan type, available benefits, exclusions, and corresponding values; and tabular templates that include descriptions structured like database rows/records with different columns for benefit attributes.

In some embodiments, parsing the natural language question includes using a cache to store frequently asked questions, and in accordance with a determination that the cache stores the natural language question, applying semantic search on the cache to identify the user intent.

In some embodiments, the method further includes in accordance with a determination that the cache does not store the natural language question, applying natural language parsing to identify the user intent.

In some embodiments, the user intent is represented by a benefit service, a location of service, and provider network status, wherein the user intent is used to retrieve context from a structured database.

In some embodiments, the recommendation algorithm is trained to rank benefit services based on features including semantic similarity and static features including benefit coverage and benefit cost.

In some embodiments, the recommendation algorithm ranks a benefit with a lower cost higher than a benefit with a higher cost if both benefits are covered.

In some embodiments, the method further includes testing and updating the recommendation algorithm and/or the large language model based on determining if the response includes specific values for copayment for the benefit.

In some embodiments, the natural language question includes a question concerning a topic selected from the group consisting of: member eligibility, benefit coverage, cost of benefit, healthcare cost accumulation, a setting or location for a benefit, visit limits or dollar maximums, and prior authorization requirement for receiving a benefit, specific providers, facility or professional credentials needed, provider in or out of network, specific diagnoses, procedures, and experimental or investigational restrictions.

In another aspect, a computer system includes one or more processors, memory, and one or more programs stored in the memory. The programs are configured for execution by the one or more processors. The programs include instructions for performing any of the methods described herein.

In another aspect, a non-transitory computer readable storage medium stores one or more programs configured for execution by one or more processors of a computer system. The programs include instructions for performing any of the methods described herein.

### BRIEF DESCRIPTION OF THE DRAWINGS

FIG. 1 is a schematic diagram of an example system for real-time and diagnostic omnichannel interaction insights, actions, and management using machine learning models, according to some embodiments.

FIG. 2 is a schematic diagram of an example system for providing real-time diagnostic interaction insights, according to some embodiments.

FIGS. 3A-3D show a schematic diagram of an example system for enhanced domain-specific analytics, according to some embodiments.

FIG. 4 is a schematic diagram of an example system for AI-based service quality auditing, according to some embodiments.

FIG. 5A shows an example cognitive platform user interface for surfacing reports and/or analytics generated using techniques described herein, according to some embodiments.

FIG. 5B shows another example cognitive platform user interface for surfacing reports and/or analytics generated using techniques described herein, according to some embodiments.

FIG. 5C shows another example cognitive platform user interface for surfacing reports and/or analytics generated using techniques described herein, according to some embodiments.

FIG. 6 is a flowchart of an example method for generating real-time and diagnostic omnichannel interaction insights, according to some embodiments.

FIG. 7 is a flowchart of an example method for generating enhanced domain-specific analytics, according to some embodiments.

FIGS. 8A and 8B show example conversational interfaces, according to some embodiments

Like reference numerals refer to corresponding parts throughout the drawings.

### DESCRIPTION OF IMPLEMENTATIONS

Reference will now be made to various implementations, examples of which are illustrated in the accompanying drawings. In the following detailed description, numerous specific details are set forth in order to provide a thorough understanding of the invention and the described implementations. However, the invention may be practiced without these specific details. In other instances, well-known methods, procedures, components, and circuits have not been described in detail so as not to unnecessarily obscure aspects of the implementations.

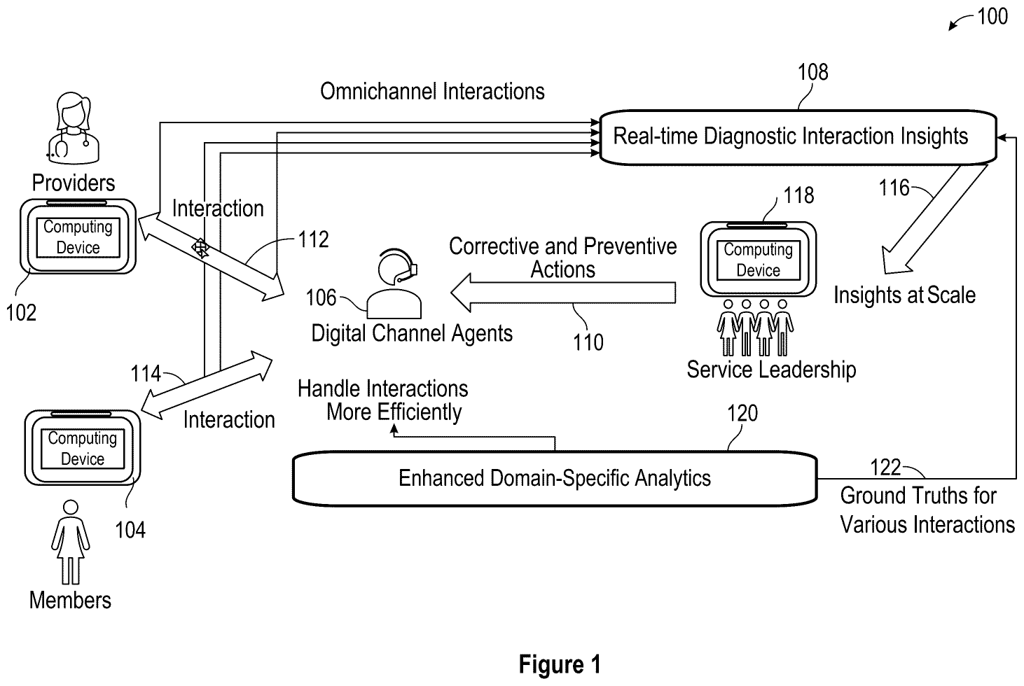

Disclosed embodiments enable generation of real-time and/or diagnostic omnichannel interaction insights, and/or generation of enhanced domain-specific analytics. Systems, methods and devices implementing the techniques in accordance with some embodiments are illustrated in FIGS. 1-7.

As described in the Background section, omnichannel interactions provide significant advantages. In healthcare, health payors may offer consumers various health plans, products and/or programs. Healthcare ecosystem products may be complex in nature and require a nuanced understanding of the product structure, benefits, and/or use. For instance, health insurance products have benefits or services that are covered, networks and providers, cost share elements that may be unique to a plan (e.g., deductible, out of pocket maximum). As users have questions about the products or services they purchased, there may be several service channels (sometimes referred to as digital channels, may include analog) for the users to reach out to a digital channel agent (e.g., an agent servicing a health insurance plan) to ask questions. For example, questions may include what is included within a product, how to leverage a service, how services were rendered, and so on. Health payors may have millions of members and may receive tens of millions of phone calls and digital interactions quarterly, ranging from simple questions on whether a benefit is covered, to clinical oncology programs, to how to pay a premium.

Some embodiments provide visibility into a broad scope of interactions of members have with the system (e.g., via chat, phone, mobile applications, web applications, through third party applications). Insights and feedback may be provided automatically instead of, or in addition to, manual review, listening and analysis of interaction, to identify any opportunities for engaging more empathetically with members/providers, and/or for providing more accurate information and answers to stakeholders. Some embodiments understand trending insights and identify issues through the existence of key words and phrases uttered on phone call or typed in chats. For instance, if members utter competitor names, there may be a potential for a member loss. As another example, if members indicate that issues are not resolved, some actions may be performed for service recovery. The sampling nature of reviewing interactions may cause specific issues to be overlooked.

FIG. 1 is a schematic diagram of an example system 100 for real-time and diagnostic omnichannel interaction insights, actions, and management using machine learning models, according to some embodiments. Providers (e.g., computing devices 102) interact with digital channel agents 106 via channels 112. Similarly, members (e.g., computing devices 104) interact with the digital channel agents 106 via channels 114. Some embodiments provide enhanced domain-specific analytics 120 to answer queries from the digital channel agents 106 to support stakeholders (e.g., the computing devices 102 and the computing devices 104) across interactions, such as benefits questions, claims processing questions. Some embodiments provide real-time diagnostic interaction insights 108 based on omnichannel interactions (e.g., interactions via the channels 112 and 114). The insights and/or analytics may be generated using large language models and other techniques described below. The real-time diagnostic interaction insights 108 may be used to generate insights at scale 116 to service leadership (e.g., computing devices 118) that monitor and/or manage performance of services via the digital channels 112 and 114. The insights 116 may include results and/or surface trends and issues at scale in near real-time. Some embodiments provide an interface/application to surface the content in a meaningful way to the digital channel agents 106 and/or computing devices 118, 102 and/or 104, to enable systemic corrective and/or preventive actions 110. Some embodiments use natural language processing. The domain-specific analytics 120 may provide ground truth 122 for generating the interaction insights 108, according to some embodiments. Example systems, algorithms, methods and techniques for implementing the enhanced domain-specific analytics 120 and the real-time diagnostic interaction insights 108 are further described below. The domain-specific analytics 120 and the real-time diagnostic interaction insights 108 may be implemented in a single system or separate systems (to operate independently). Furthermore, the domain-specific analytics 120 may be implemented in an online mode (e.g., to operate in real-time or near real-time) and/or in an offline mode (e.g., to analyze benefit documents and/or inquiries and provide analytics in a batch mode or at a periodic schedule, e.g., hourly, daily, weekly).

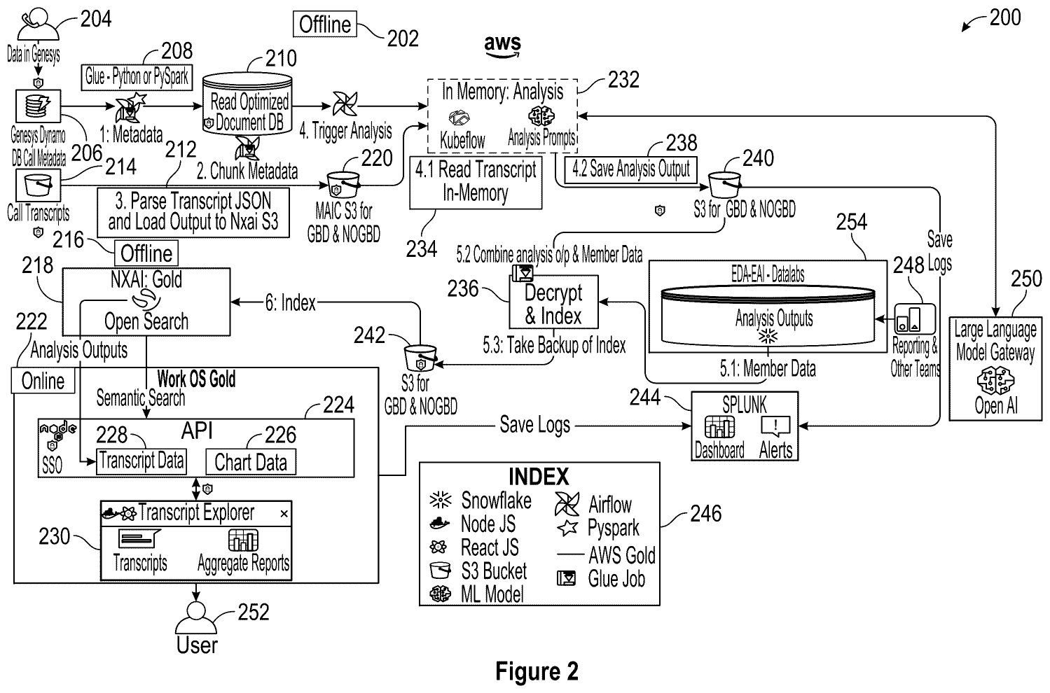

FIG. 2 is a schematic diagram of an example system 200 for providing real-time diagnostic interaction insights 108, according to some embodiments. Some embodiments provide channel data integration (e.g., call data integration). Some embodiments interface with telephonic operations and call recording, and/or transcription services. The transcripts may be accessible through an API. Due to API call limitations, the data may be staged for organizational consumption. Some embodiments provide a metadata storage. Call-associated metadata may be cataloged and stored in a database, facilitated by a call center platform. Some embodiments provide a transcripts storage 214 (e.g., AWS S3). In some embodiments, a call center platform interfaces with a provider to retrieve call transcripts. The transcripts may be subsequently stored in the transcript storage 214. For example, data provided by Genesys 204 may be retrieved using a Genesys Dynamo API 206, to obtain call transcripts and/or call metadata. Glue code 208 (e.g., Python or PySpark code) may be used to retrieve and/or extract metadata into a read-optimized document database 210. Some embodiments use a document-oriented database cache. To optimize transcript processing, a read-optimized document database may be used as an intermediary cache.

Reading the metadata 210 may trigger in-memory analysis 232, which may use Kubeflow and/or analysis prompts, and include reading transcripts in-memory (234) and saving or storing analysis output (238) (e.g., to a S3 storage 240). An hourly ETL job, which may be orchestrated via Airflow, may activate either a transient EMR cluster or an AWS Glue job. This operation may identify and/or and migrate new records into a database. Some embodiments provide in-memory transcript analysis. In some embodiments, upon metadata availability, a Kubeflow-based job may be initiated, deploying multiple pods. Each pod may process transcript segments, leveraging metadata from the document database and/or the transcript from the call center platform's storage (e.g., S3). Machine Learning models may be applied for in-depth transcript analysis.

Reporting and/or management systems 248 may provide member data 254, which may include user interaction data (e.g., selection and/or deselection of user interface affordances, time spent on landing pages), efficiency and/or utilization (e.g., time computing resources are consumed, time taken to respond to events, such as user queries), and/or user feedback (e.g., thumbs up and/or thumbs down for responses to queries). The system may combine the member data 254 with the analysis output 240 to output data 236 which may be decrypted and/or indexed. The data 236 may be stored in an S3 storage 242. Some embodiments provide encrypted storage (e.g., encrypted S3 Storage). In some embodiments, post-analysis, Kubeflow pods may save the output in S3 buckets as encrypted files, utilizing 256 AES encryption. The data stored in the storage 242 may be indexed for use in an offline mode 216. The indexed data may also be searched using a search API 218 (e.g., OpenSearch API). Some embodiments provide data enrichment and/or indexing. Following the analysis phase, an AWS Glue job may be launched to integrate the analysis output with member-specific data from the Compass Snowflake database. The enriched data may be indexed within the OpenSearch instance. For redundancy, encrypted backups of this data may be maintained in the S3 store 242.

In an online mode 222, the search API 218 may be used to perform semantic search to obtain chart data 226. The search API 218 may also be used to obtain transcript data 228 based on analysis output. Some embodiments provide an OpenSearch database, which may serve as a search and analytics repositor. OpenSearch may index analysis outputs and transcripts chronologically. Real-time querying of analysis results may be facilitated for frontend systems. The transcript data 228 and the chart data 226 may be accessible via a single sign-on secure application 224. Some embodiments provide a platform to support scalable APIs and websites exclusive to a portion of a company or team. Some embodiments provide a transcript API (e.g., using Python Flask and hosted on the platform), which retrieves transcripts and/or analysis from OpenSearch., and/or a filter API (e.g., an API]implemented in Node.js), which interprets UI filters to query OpenSearch dynamically. Some embodiments provide a transcript explorer user interface 230. The user interface may be hosted on the platform. A transcript explorer 230 may provide analytical insights into trends (e.g., call traffic trends). Features may include sentiment analysis and data filtering by multiple parameters, such as time, topic, and sentiment. Data visualization may be subject to a predetermined latency (e.g., 2-day latency). A transcript explorer 230 may generate and/or display, for a user 252, transcripts and/or aggregate reports 230 based on the transcript data 228 and/or the chart data 226. Some embodiments provide a large language model gateway (e.g., OpenAI interface) for generating text based on the output of the in-memory analysis 232. Some embodiments provide dashboards/or alerts 244 (e.g., applications implemented using software for searching, monitoring, and/or analyzing machine-generated data via a web-style interface, such as Splunk) based on the transcript data 228, the chart data 226, and/or data stored in the storage 238. Index 246 shows various components, interfaces or tools that may be used in the system 200. Such components, interfaces, or tools may include Snowflake, Node JS, React JS, S3 Bucket, machine learning models, Airflow, Pyspark, AWS Gold and Glue job, and/or similar third-party software.

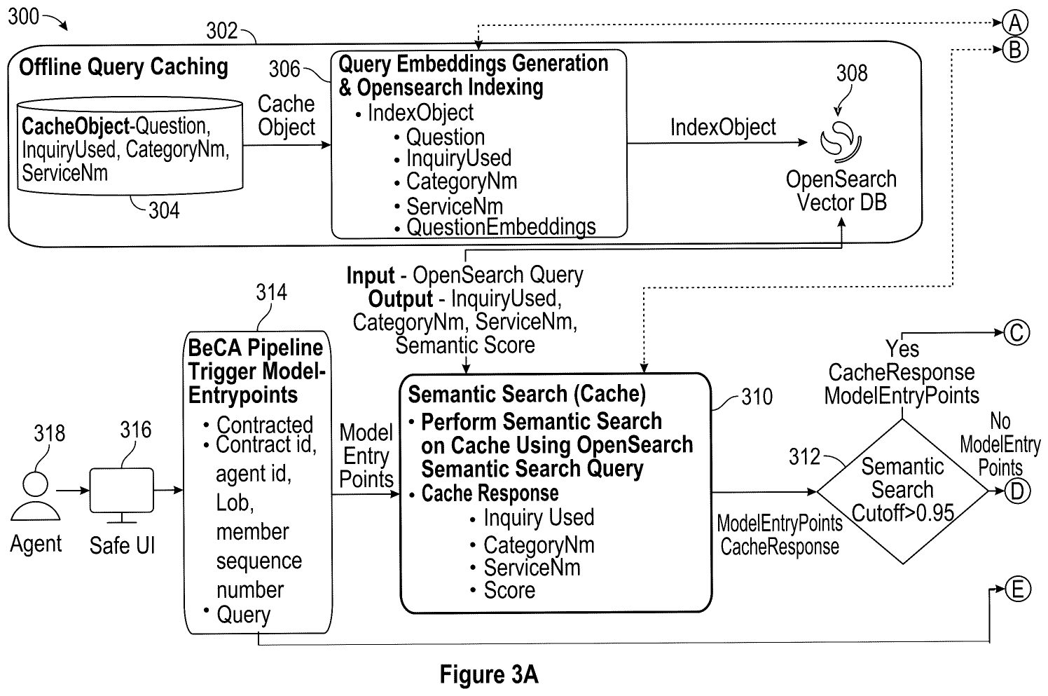

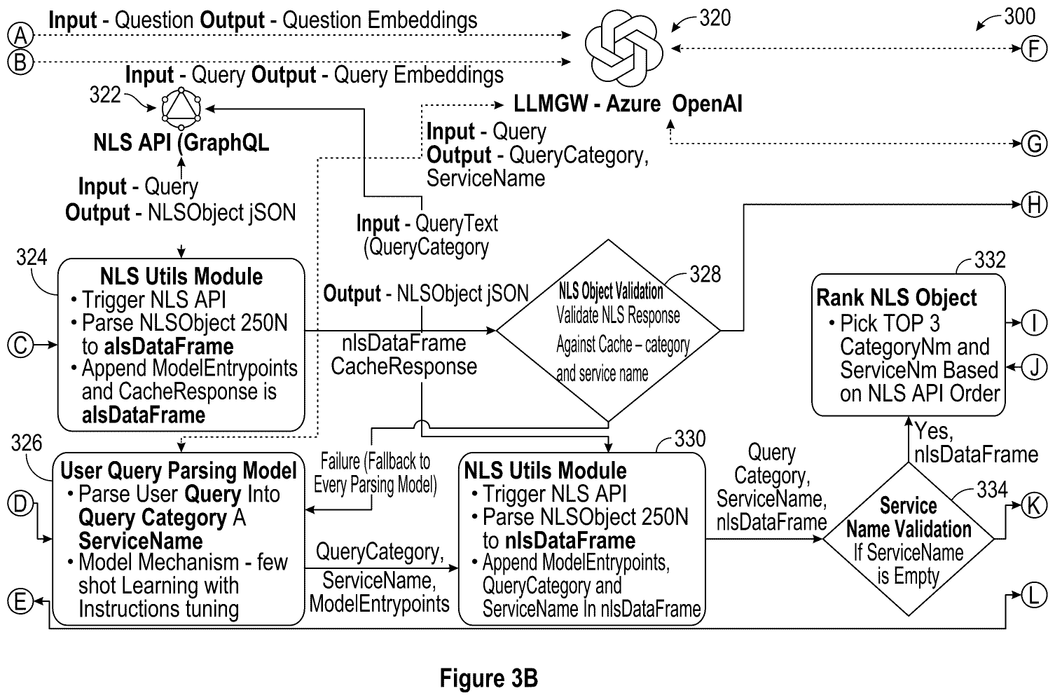

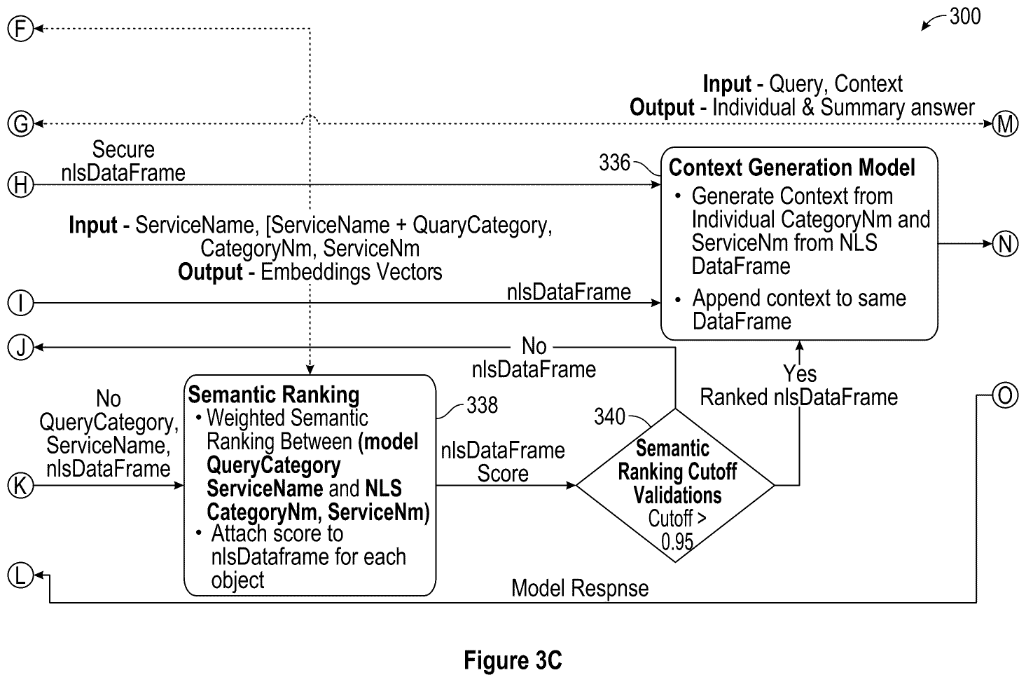

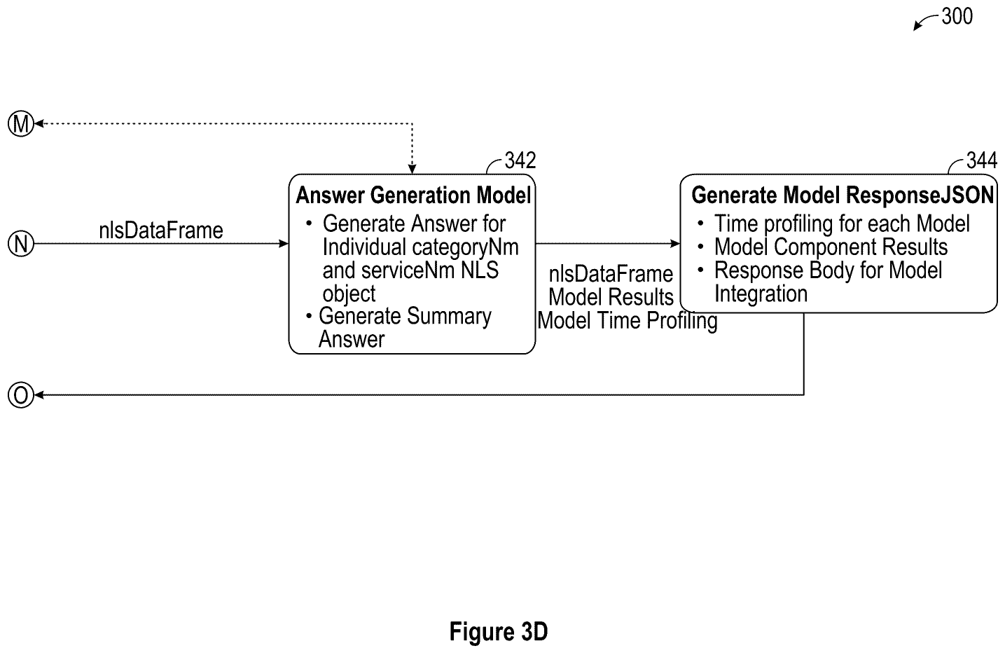

FIGS. 3A-3D show a schematic diagram of an example system 300 for enhanced domain-specific analytics 120, according to some embodiments. Some embodiments include an offline query cache 302 for caching query objects 304, which may include question, inquiry used, category number and/or service number. Cached objects from the store 304 may be provided to a module 306 for query embedding generation and/or OpenSearch indexing, which generates and/or indexing cached objects, to obtain question, inquiry used, category number, service number and/or question embeddings. Index objects from the module 306 may be input to a vector database 308 (e.g., OpenSearch VectorDB). Inputting a query (e.g., an OpenSearch query) to the vector database 308 may output an inquiry used, category number, service number, which may be used to perform a semantic search 310 to output a semantic score. Inquiries related to benefits of members, for example, may be linked to a predetermined collection of benefit categories for semantic comparison. Model processing may start or be triggered from model entry points 314. Input by an agent (e.g., contract identifier, agent identifier, Lob, interaction identifier, member sequence number), which may be input via a user interface 316, may be used for the semantic search 310. OpenSearch's semantic search query may be used for the. Some embodiments perform semantic search on a cached set of frequently asked questions. Some embodiments maintain a pool of frequently asked questions (e.g., in the cache 304) and retrieve answers when the questions in the pool semantically match a user benefit inquiry. This may help ensure accuracy for a pool of frequently asked questions. A search database, such as OpenSearch, may be used to maintain the pool of questions. The database may be scalable because it is used for database storage, instead of, or in addition to, in-memory processing. The database may be designed specifically to optimize semantic search on a large dataset. Model entry points and semantic score from the semantic search 310 may be subject to a cutoff 312 (e.g., if the semantic score is greater than 0.95).

Referring next to FIG. 3B, if the semantic score does not meet the cutoff, then there are no model entry points (label D), so a user query parsing model 326 is executed. The model may include few shot learning with instruction tuning. User query may be used to output category and service name. Some embodiments use a user query parsing model. Some embodiments use an artificial intelligence (AI) model to parse a natural language question to determine intent. In this case, the intent may be represented by a benefit service, such as immunization, pap test, durable medical equipment, location of service, such as inpatient or outpatient, and/or provider network status, such as in network or out of network. The intent may be used to retrieve context from a structured database. If the semantic score is above the cutoff, then cached response and model entry points may be sent (label C) to a natural language search (NLS) utilities module 324, which may trigger an NLS API using an NLS object and/or model entry points. Some embodiments use a large language model (LLM) to process a data source. Some embodiments pull benefit content production API. This design allows scalability. Some embodiments include one or more APIs to enrich retrieved context to help generate accurate and complete answer. The output of the module 326 may be input to an NLS utilities module 330 which may trigger an NLS API, to generate an NLS object to an NLS data frame, append model entry points, query category and service name in NLS data frame, to output a query category service name and/or an NLS data frame. An NLS API 322 (e.g., Graph QL) may be used to input a query and output an NLS object (e.g., JSON format) for the NLS utilities module 324 and/or the NLS utilities module 330. An LLM API 320 (e.g., LLMGW from OpenAI) may be used to input a query to output a query category and/or service name for the user query parsing model 326. The NLS data frame and/or the cache response output by the NLS utilities module 324 is input to an NLS object validation 328, which updates NLS and/or validates cache response and/or service name. Query category, service name, and/or NLS data frame output from the NLS utilities module 330 may be input to a service name validation 334 to determine if service name is empty. Some embodiments may include natural language support for object validation, to validate natural language response against known elements. For example, object validation may include determination if there are no services and/or benefits for a member, determination if the benefits have been carved out to be delivered by another company, and so on.

If the service name is validated, an NLS data frame is input to rank NLS object 332, which picks a predetermined top number (e.g., top 3) category name, service name based on an NLS API order. Some embodiments perform benefit service ranking. Some embodiments include an AI module to rank retrieved benefit content from production API. Learning to rank may be a type of recommendation algorithm that ranks benefits. For example, if the user intent is to look up benefit of immunization, the system may rank flu shot higher than other benefits because it is the most relevant benefit. The ranking model may use features, such as semantic similarity, and static features, such as benefit coverage. Some embodiments rank covered relevant benefit above other types of benefits. Cost of the service may also be a feature. For similar covered services, some embodiments rank the benefit based on the cost.

If the service name is not validated, no query category, service name, and/or NLS data frame may be available and/or input to semantic ranking 338, which weights semantic ranking between model query category, service name and NLS category name, service name. Service name, query category, category name, and/or service name may be input to the LLMGW 320 to output embedding vectors for the semantic ranking 338. The rank score may be attached to an NLS data frame for each object and input to a semantic ranking cutoff validations 340 (e.g., cutoff greater than 0.95). Ranked NLS dataframe, if the threshold is met, may be input to a context generation model 336. If the cutoff threshold is not met, no NLS data frame is input to the rank NLS object 332 (FIG. 3B).

In some embodiments, the benefits may be in a format (e.g., JSON), which may not be input to a large language model. Accordingly, some embodiments include the context generation model 336 to compose context into natural language using language templates. The context generation model 336 may generate context from individual category name, service name from NLS data frame, and/or append context to the same data frame, based on secure NLS data frame from the NLS object validation 328, NLS data frame from the rank NLS object 332, and/or ranked NLS data frame from the semantic ranking 338.

Some embodiments include an LLM answer generation model 342, which may be invoked to process answer from a generated context. The LLM model may take benefit content as context and summarize answer from the context. For answer completeness, some embodiments insert specific rules in a prompt to cover different perspectives in benefit. For example, if cost shares (e.g., deductible, coinsurance, copayment) are same for different services/locations, a rule may be to state them together. As another example, suppose a copayment or coinsurance of $0 is the same as “no copayment” or “no coinsurance” respectively. A rule may be to use the latter language (“no copayment/coinsurance”). To reduce hallucination, some embodiments may also insert a knowledge base into the prompt to enrich concepts that can be understood by the model. Query and/or context may be input to the LLMGW 320 to output individual and/or summary answer for the LLM generation model 342. NLS data frame from the context generation model 336 may be input to the answer generation model 342. The answer generation model 342 may output an NLS data frame, model results and/or model time to generate model response 344 (e.g., in a JSON format), which may output time for each model, model component results, and/or response body for model integration, and/or maybe input to the pipeline trigger model entry points 314.

Some embodiments use human in the loop testing. Some embodiments provide a user interface to support review of output from the LLMs by subject matter experts. Some embodiments allow uploading test cases which shows context, question and answer to UI for display. Subject matter experts can tag agree, partially agree and disagree to use cases. This enables the system to be accurate and accommodate feedback efficiently.

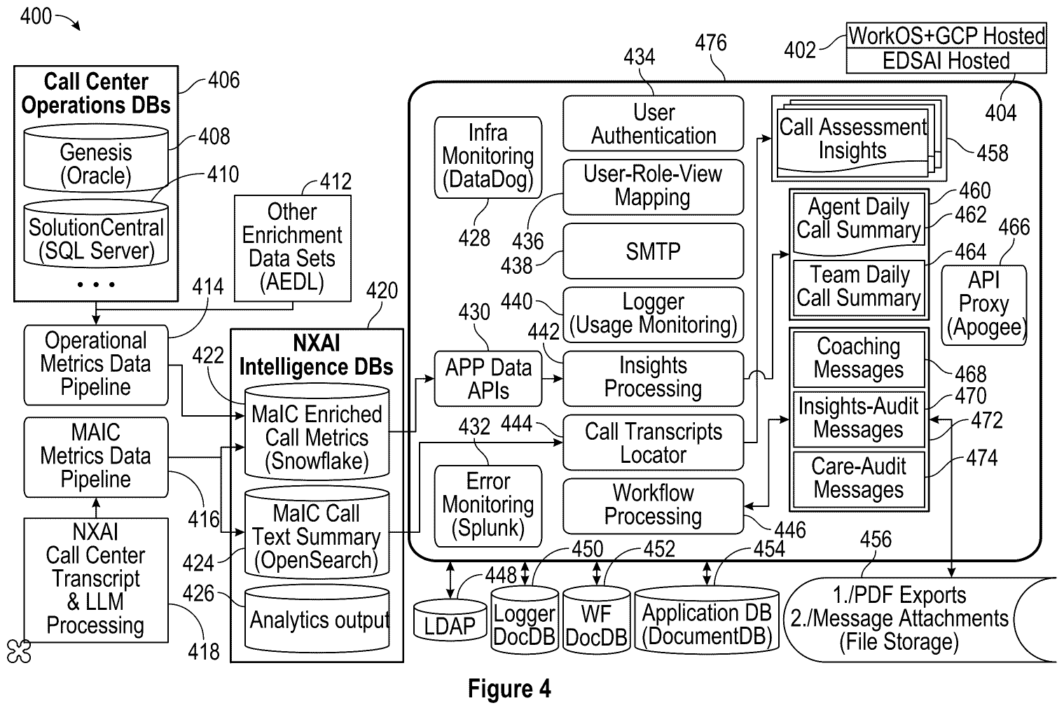

FIG. 4 is a schematic diagram of an example system 400 for AI-based service quality auditing, according to some embodiments. Call center operations databases 406 may include a Genesis (or a similar) database 408, and/or a solution central 410 (e.g., an SQL server), which may supply data to an operational metric data pipeline 414, which may receive input from other enrichment data sets 412. Transcripts (e.g., NXAI call center transcripts) and/or LLM processing 418 may be input to a metrics data pipeline 416 (e.g., the real-time diagnostic interaction insights 108) whose output and/or the output from the operational metrics data pipeline 416 (e.g., the enhanced domain-specific analytics 120) may be stored in intelligent databases 422 (e.g., NXAI databases), which may include enriched metrics 422 (e.g., call metrics), text summary 416 (e.g., call text summary), and/or analytics output 426 (e.g., store output of the enhanced domain-specific analytics 120). Service quality auditing 476 may include an infrastructure monitoring 428 (e.g., implemented using DataDog), user authentication 434, user-role-view mapping 436, SMTP 438, logger (usage monitoring) 440, error monitoring 432 (Splunk), and/or API proxy 466 (e.g., Apogee). Enriched metrics 422 may be input to an application data APIs 430, which may output data to insights processing 442. Text summary 424 may be input a call transcripts locator 444. Output from the call transcripts locator 444 may be input to a call assessment insights 458. Output from the insights processing 442 may be input to an agent daily call summary 460 and/or team daily call summary 464, to form a call summary 460. Output from a workflow processing 446 may be input to coaching messages 468, insights-audit messages 470, and/or care-audit messages 474, to form messages 472. The messages 472 may be input to PDF exports and/or message attachments 456 (file storage). The audits may also be stored or interface with an application database 454 (e.g., Document DB), a workflow document database 452, a logger document database 450, and/or LDAP 448. The system 400 may be implemented using WorkOS, hosted in a cloud (e.g., GCP), and/or EDSAI hosted 402.

### Example Insights Applications and User Interfaces

Some embodiments provide an interface to view output from one or more machine learning models described herein. Output may include summaries, sentiment, and/or notation of follow ups or promises. In some embodiments, output visualization may be presented to users in a meaningful way for systemic action to improve overall service. For example, visualizations may include identification of microtrends within populations that are actionable (e.g., respond to provider availability challenges, medical device company supply chain issues, by adjusting providers, supply schedules, and so on).

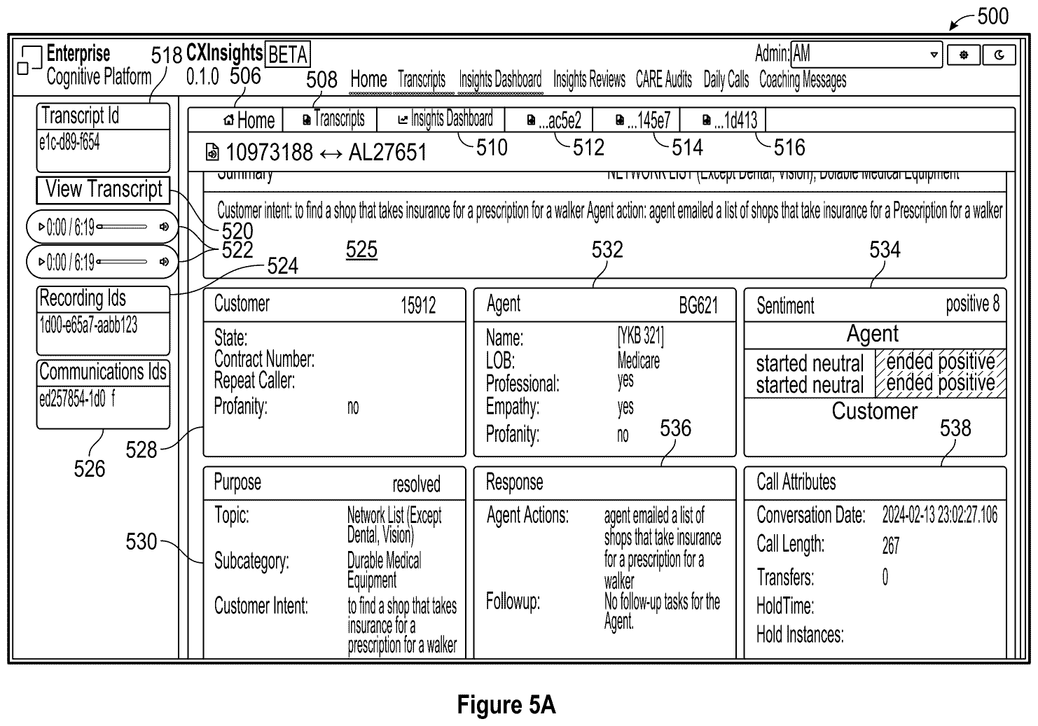

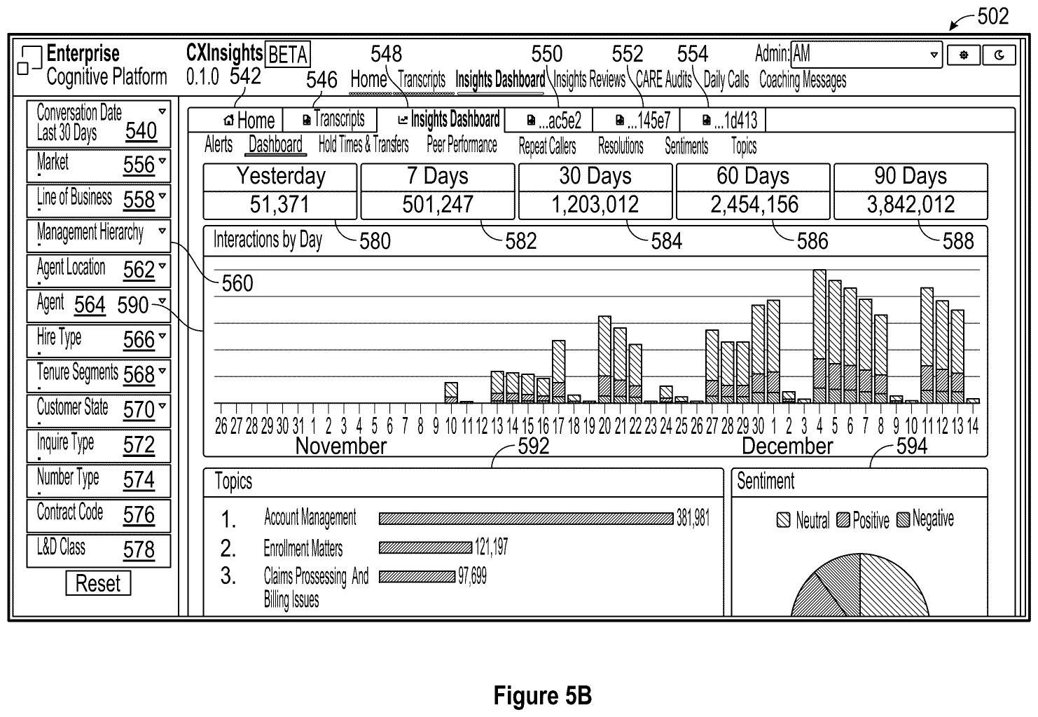

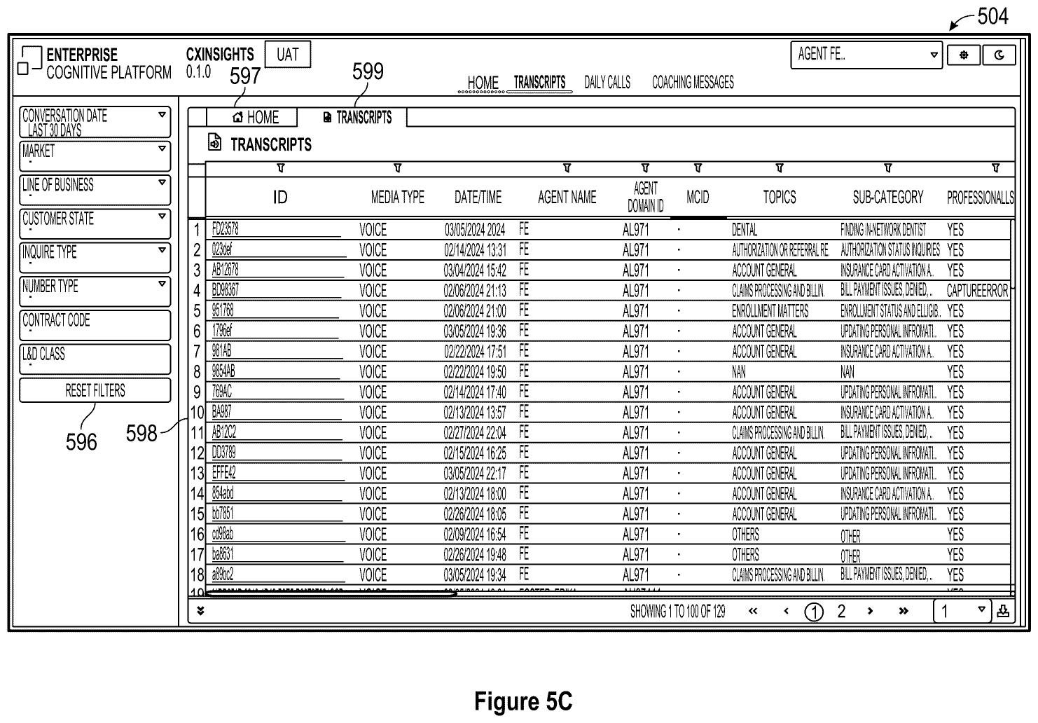

FIG. 5A shows an example cognitive platform user interface 500 (sometimes referred to as a dashboard) for surfacing reports and/or analytics generated using techniques described herein, according to some embodiments. The dashboard may show a transcript identifier 518, an affordance 520 to view transcript, one or more call affordances (e.g., affordance 522), recording identifiers 524, and/or communications identifiers 526. A home button 506, affordances for transcripts 508, insights dashboard 510, and/or insights or transcripts affordances (e.g., affordances 512, 514, 516) may be shown. A region 525 may show a summary of user intent and any action that may have been taken for user intent. Customer details 528, agent details 532, sentiment 534, purpose 530, response 536, and/or call attributes 538 may also be shown, according to some embodiments.

FIG. 5B shows another example cognitive platform user interface 502 for surfacing reports and/or analytics generated using techniques described herein, according to some embodiments. Affordances 542, 546, 548, 550, 552, 554 are similar to corresponding affordances in FIG. 5A. Conversation date 540, market 556, line of business 558, management hierarchy 560, agent location 562, agent 564, hire type 566, tenure segments 568, customer state 570, inquire type 572, number type 574, contract code 576, and/or class identifier 578, may be shown in a region of the user interface. The dashboard may number of calls/interactions over a different periods of time may be shown in a different region of the user interface (e.g., interactions encountered yesterday 580, 7 days 582, 30 days 584, 60 days 586, and 90 days 588). A bar chart 590 may show interactions by day. Top topics (e.g., top 3 topics) for the interactions 592 may be shown separately. A sentiment (e.g., neutral, positive, or negative) may be shown separately (e.g., as a pie chart 594).

FIG. 5C shows another example cognitive platform user interface 504 for surfacing reports and/or analytics generated using techniques described herein, according to some embodiments. Affordances on the left are similar to affordances for conversation date 540, market 556, line of business 558, management hierarchy 560, agent location 562, agent 564, hire type 566, tenure segments 568, customer state 570, inquire type 572, number type 574, contract code 576, and/or class identifier 578, of FIG. 5B, which may be shown in a left region of the user interface. A reset filter 596 allows users to reset any filters applied to the dashboard. A home tab 597 and/or transcripts tab 599 may be shown in another region of the user interface. Selecting the transcripts tab may show a list of all transcripts (e.g., identifier, media type, such as voice, video, text, date/time agent name, topics, subcategory, and/or evaluations, such as errors, resolutions).

Some embodiments provide a closed loop feedback system that continuously improves the solutions described above.

The techniques described herein may be implemented in a servicing platform and/or workflow system. The systems, methods and interfaces may be implemented in a payor ecosystem and/or for member/stakeholder interaction analysis. The techniques described herein may be used in scenarios in which customers are interacting with a system at scale through multi-variable channels. These techniques help unify a set of solutions to enable consistent answers, insights at scale.

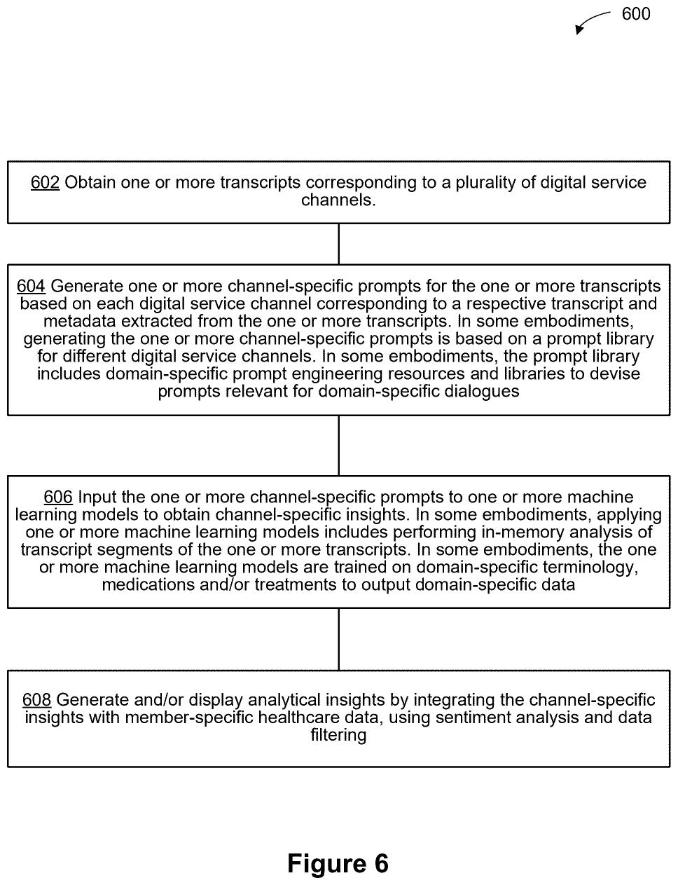

FIG. 6 is a flowchart of an example method 600 for generating real-time and/or diagnostic omnichannel interaction insights, according to some embodiments. The method may be performed by the real-time diagnostic interaction insights 108, FIG. 1, an example of which is described above in detail in reference to FIG. 2. The method may include obtaining (602) one or more transcripts corresponding to a plurality of digital service channels. The plurality of digital service channels may include two or more channels selected from the group consisting of: (i) a phone channel for interaction with agents trained to respond about benefits, claims and providers; (ii) an email or secure messaging channel for written inquiries about benefits, claims, healthcare documents, including attachments for evidence of claim, explanation of benefits statements; (iii) a chat or instant messaging channel for real-time interaction to obtain domain related information; (iv) a web portal channel for secure online accounts to view benefits, check claim status, order identifier cards, updating contact information, uploading claims and documents; and (v) a social media channel for responding to public inquiries, providing updates during events impacting members. In some embodiments, the one or more transcripts include: (i) text related to healthcare topics including claims, benefits, insurance plans, coverage, medical terminology, and regulations; (ii) at least some data with protected health information; (iii) communication between patients, insurance companies and healthcare providers; and/or (iv) speech and text data, including telephonic operations and call recordings. In some embodiments, obtaining the one or more transcripts includes interfacing with one or more third-party provider computers (e.g., data in Genesys 204), to receive text and/or speech data. In some embodiments, obtaining the one or more transcripts includes optimizing transcript processing using a read-optimized document database (e.g., the database 210) as an intermediary cache. An hourly ETL job, orchestrated via Airflow, may activate either a transient EMR cluster or an AWS Glue job, thereby identifying and migrating new records into a database. In some embodiments, the one or more transcripts combines text or speech data obtained from the plurality of health service channels. At least two of the plurality of health service channels may generate text or speech in distinct format or structure. In some embodiments, the one or more transcripts include text that is protected health information that is anonymized and/or is compliant with HIPAA and/or privacy regulations. In some embodiments, the transcript includes labeled speakers at least one of who is a healthcare service agent and another is a member of a healthcare service.

The method may also include generating (604) one or more channel-specific prompts (e.g., the analysis prompts in the in-memory analysis 232, FIG. 2) for the one or more transcripts based on each digital service channel corresponding to a respective transcript and metadata extracted from the one or more transcripts. Generating the one or more channel-specific prompts may be based on a prompt library for different digital service channels. The prompt library may include domain-specific prompt engineering resources and libraries to devise prompts relevant for domain-specific dialogues. Generating the one or more channel-specific prompts may be based on: analyzing common queries or intents in domain-specific (e.g., healthcare-specific) omnichannel interaction data to identify frequently occurring query patterns, intents, and/or topics that domain users express; using intent classification on domain-specific omnichannel interaction data to categorize utterances into distinct buckets like benefits inquiry, claims assistance and provider search to use intent categories to generate prompts; using named entity recognition (NER) to extract entities like medication names, treatment procedures, and insurance terms, in healthcare omnichannel interaction data to frame prompts incorporating the entities; analyzing and/or reverse engineering prompt-response pairs from prior domain-specific omnichannel interaction data to discern patterns and templates for new prompts; using input from domain experts like physicians, nurses and claims specialists to use domain knowledge to suggest prompts spanning different healthcare scenarios and contexts; and/or performing A/B tests with candidate prompts with a large language model to assess response quality, clarity, specificity and adherence to healthcare compliance, to iteratively refine prompts based on test results.

The method may also include inputting (606) the one or more channel-specific prompts to one or more machine learning models to obtain channel-specific insights. Applying the one or more machine learning models may include performing in-memory analysis (e.g., the in-memory analysis 232) of transcript segments of the one or more transcripts. The one or more machine learning models may be trained on domain-specific terminology, medications and/or treatments to output domain-specific data. In some embodiments, the method includes storing the one or more transcripts in a cloud object storage (e.g., the storage 240), and/or cataloging and/or storing the metadata in a NoSQL database or persistent key-value datastore for replication, autoscaling, encryption at test, and on-demand backup. In some embodiments, performing the in-memory analysis includes, upon availability of the metadata, initiating a Kubeflow-based job (e.g., Kubeflow in in-memory analysis 232), deploying a plurality of pods, each pod processing transcript segments, based on metadata from a document database and the one or more transcripts. In some embodiments, the method further includes storing, by the plurality of pods, the channel-specific insights in a cloud object storage as encrypted files, using 256 AES encryption. In some embodiments, the one or more machine learning models are trained to identify healthcare related topics and/or sub-topics within each transcript. In some embodiments, the one or more machine learning models are trained to identify member sentiment for each transcript, using sentiment analysis. In some embodiments, the one or more machine learning models are trained to generate a summary for each transcript or the one or more transcripts.

The method may also include generating and/or displaying (608) analytical insights by integrating the channel-specific insights with member-specific healthcare data, using sentiment analysis and data filtering. This is illustrated as step 5.2 in FIG. 2. In some embodiments, applying data filtering includes filtering the call-specific insights and the member-specific data by a plurality of parameters, including time, topic, and/or sentiment. In some embodiments, the method further includes generating a data visualization (e.g., the graphical user interfaces or dashboards described above in reference to FIGS. 5A, 5B, or 5C) based on the analytical insights, the data visualization subject to a predetermined latency. In some embodiments, the method further includes indexing the analytical insights and/or the one or more transcripts chronologically for real-time querying. In some embodiments, generating the analytical insights includes integrating interactions across multiple health service channels for a same member over time. For example, a member (e.g., a computing device 104 corresponding to a member) interacts with multiple digital channel agents 106 over time. In some embodiments, generating the analytical insights includes generating an issue resolution flag for a transcript to indicate if an issue raised in the transcript has been resolved in an interaction corresponding to the transcript. In some embodiments, generating the analytical insights includes identifying a unique member identification for healthcare service members. For example, a member may be assigned a unique member identifier across different providers.

In some embodiments, the method further includes providing one or more application programming interfaces (APIs) (e.g., the APIs 224) for (i) retrieving and/or (ii) interpreting user interface filters to query, the one or more call transcripts, the call-specific insights, and/or the analytical insights.

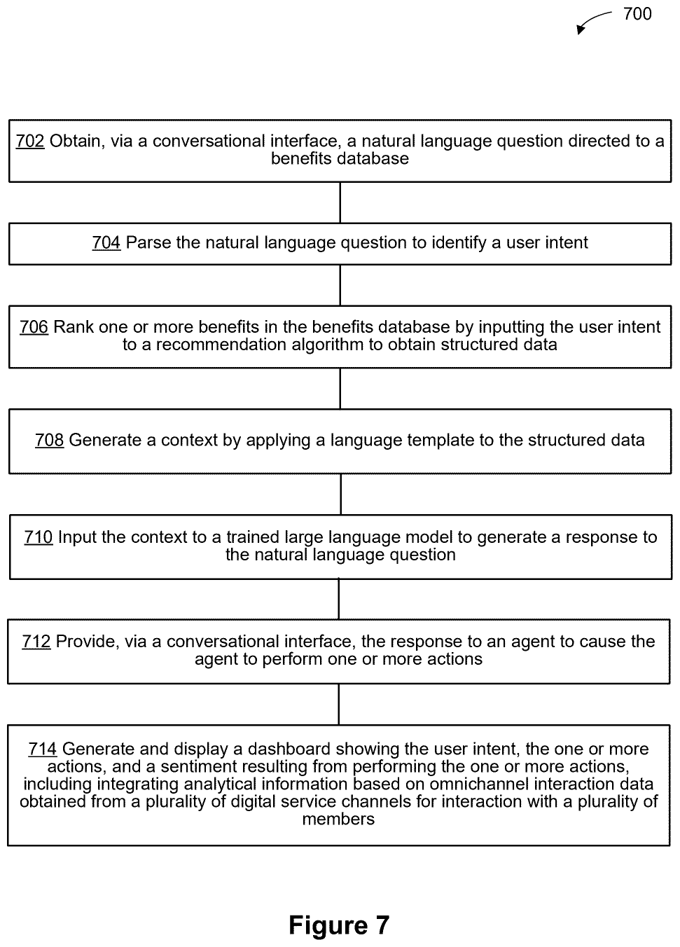

FIG. 7 is a flowchart of an example method 700 for generating enhanced domain-specific analytics, according to some embodiments. The method may be performed by the enhanced domain-specific analytics 120, FIG. 1, an example of which is described above in reference to FIGS. 3A-3D, according to some embodiments. The method may include obtaining (702), via a conversational interface, a natural language question directed to a benefits database (e.g., a benefit or a member inquiry). In some embodiments, the natural language question includes a question concerning a topic selected from the group consisting of: member eligibility, benefit coverage, cost of benefit, healthcare cost accumulation, a setting or location for a benefit, visit limits or dollar maximums, and/or prior authorization requirement for receiving a benefit, specific providers, facility or professional credentials needed, provider in or out of network, specific diagnoses, procedures, and/or experimental or investigational restrictions. A natural language question may concern topics other than (or in addition) to benefits. For example, the questions may be related to network status of a provider, status of a claim, claim denial explanation, medical policy exclusions or parameters, and so on.

The method may also include parsing (704) (e.g., by the user query parsing model 326) the natural language question to identify a user intent. The user intent may be represented by a benefit service (e.g., immunization, pap test, durable medical equipment), a location of service (e.g., inpatient/outpatient), and provider network status (e.g., in-network/out-of-network). The user intent may be used to retrieve context from a structured database.

The method may also include ranking (706) one or more benefits in the benefits database by inputting the user intent to a recommendation algorithm to obtain structured data. The recommendation algorithm (e.g., rank NLS object 332, FIG. 3B) may be learning to rank algorithm that may be trained by: preparing benefits data related to various benefits, their descriptions, coverage details, eligibility criteria, historical data on how users have searched and interacted with different benefits in the benefits database, by extracting query-benefit pairs along with relevance ratings from user interactions or expert annotations. An NLS API may serve as a primary reference for member benefit data, may provide the benefit data in real-time; extracting one or more features from the benefits data that influence ranking, such as benefit type, coverage scope, cost-sharing details, provider network, applicable conditions/treatments, query features like keyword matches, semantic similarity with benefit text, user profile signals, query and benefit attributes; and using the extracted features to train a learning to rank (LTR) model like LambdaRank, RankNet, or ListNet to learn a ranking function that optimizes for a desired metric (e.g., NDCG) by minimizing the loss between predicted and true relevance rankings for benefits. The recommendation algorithm may be trained to rank benefit services based on features including semantic similarity and static features including benefit coverage and benefit cost. Semantic similarity may measure similarity between two phrases or words based on their meaning. Semantic similarity may be used to rank relevant categories. The recommendation algorithm may rank a benefit with a lower cost higher than a benefit with a higher cost if both benefits are covered.

The method may also include generating (708) a context (e.g., by the context generation model 336) by applying a language template to the structured data. The language template may include one or more templates selected from the group consisting of: descriptive sentence templates including benefit names, coverage details, eligible conditions or treatments, plan types; question-answer templates including benefit names, coverage details, eligibility criteria, cost-sharing details; conversational templates including benefit names, plan types, conditions or treatments; structured key-value templates including benefit name, coverage, eligibility, cost, providers, plan type, available benefits, exclusions, and corresponding values; and tabular templates that include descriptions structured like database rows/records with different columns for benefit attributes;

The method may also include inputting (710) the context to a trained large language model (e.g., by the answer generation model 342 and/or the generate model response JSON 344) to generate a response to the natural language question. In some embodiments, generating the response to the natural language question further includes integrating analytical information based on omnichannel interaction data obtained from a plurality of digital service channels for interaction with a plurality of members.

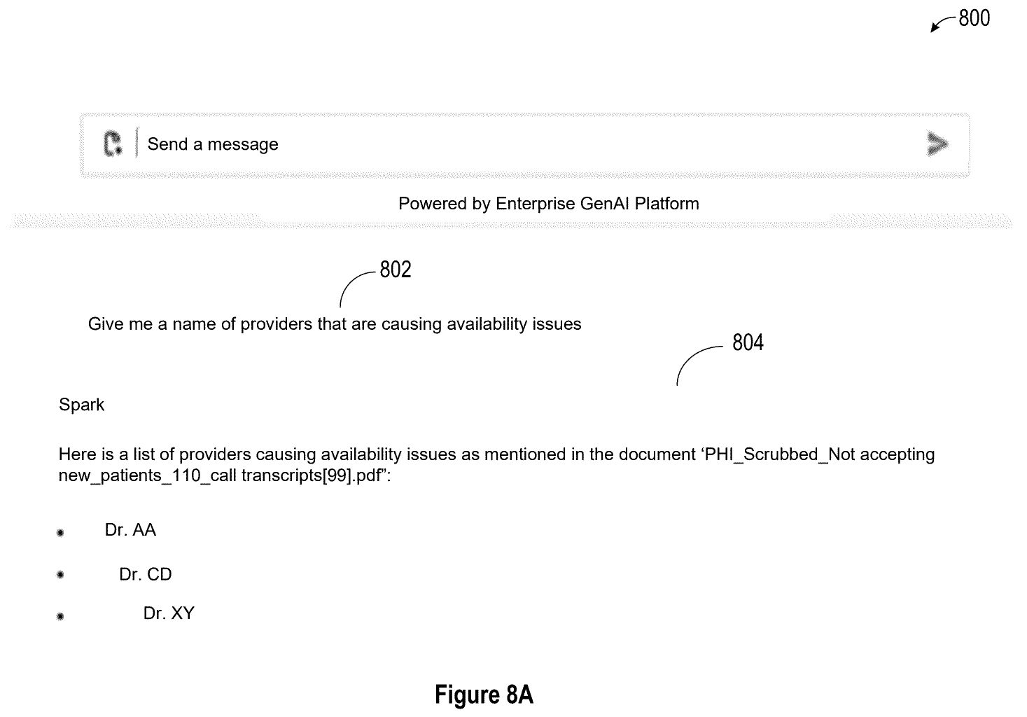

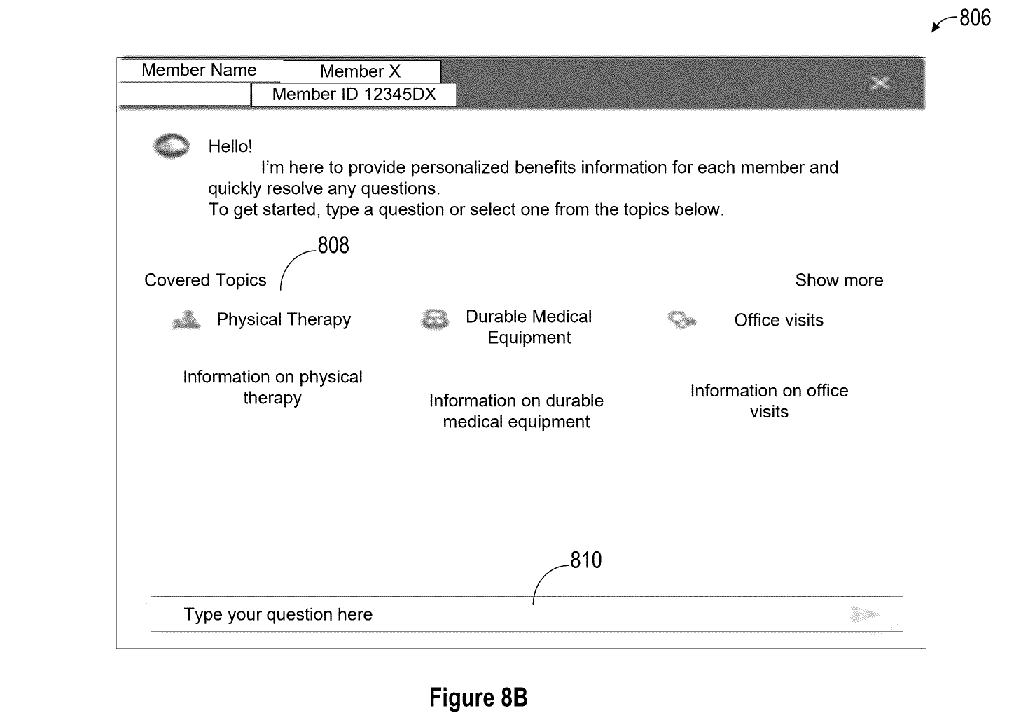

The method may also include providing (712), via a conversational interface, the response to an agent to cause the agent to perform one or more actions. FIGS. 8A and 8B show example conversational interfaces 800 and 806, respectively, according to some embodiments. In FIG. 8A shows an example interface 800 for obtaining information regarding providers. A user may enter a query, e.g., in a text box 802 (“give me a name of providers that are causing availability issues”). In response, the system may provide a list of providers 804. FIG. 8B shows an example interface 806 for providing personalized benefits information for member(s) to resolve any queries. A user may type a question (e.g., in a text box 810) or select from one of the topics 808 (e.g., physical therapy, Durable Medical Equipment, office visits).

The method may also include generating and/or displaying (714) a dashboard (e.g., user interfaces 500, 502, 504) showing the user intent, the one or more actions, and a sentiment resulting from performing the one or more actions, including integrating analytical information based on omnichannel interaction data obtained from a plurality of digital service channels for interaction with a plurality of members.

In some embodiments, the method further includes testing and updating the recommendation algorithm and/or the large language model based on determining if the response includes specific values for copayment for the benefit.

The foregoing description, for purpose of explanation, has been described with reference to specific implementations. However, the illustrative discussions above are not intended to be exhaustive or to limit the invention to the precise forms disclosed. Many modifications and variations are possible in view of the above teachings. The implementations were chosen and described in order to best explain the principles of the invention and its practical applications, to thereby enable others skilled in the art to best utilize the invention and various implementations with various modifications as are suited to the particular use contemplated.

What is claimed is:

1. A method for generating real-time and diagnostic omnichannel interaction insights, the method comprising:
obtaining one or more transcripts corresponding to a plurality of digital service channels;
generating one or more channel-specific prompts for the one or more transcripts based on each digital service channel corresponding to a respective transcript and metadata extracted from the one or more transcripts;
inputting the one or more channel-specific prompts to one or more machine learning models to obtain channel-specific insights; and
generating and displaying analytical insights by integrating the channel-specific insights with member-specific healthcare data, using sentiment analysis and data filtering.

2. The method of claim 1, wherein generating the one or more channel-specific prompts is based on a prompt library for different digital service channels, wherein the prompt library includes domain-specific prompt engineering resources and libraries to devise prompts relevant for domain-specific dialogues.

3. The method of claim 1, wherein generating the one or more channel-specific prompts is based on:
analyzing common queries or intents in domain-specific omnichannel interaction data to identify frequently occurring query patterns, intents, and topics that domain users express;
using intent classification on domain-specific omnichannel interaction data to categorize utterances into distinct buckets to use intent categories to generate prompts;
using named entity recognition (NER) to extract entities from healthcare omnichannel interaction data to frame prompts incorporating the entities;
analyzing and/or reverse engineering prompt-response pairs from prior domain-specific omnichannel interaction data to discern patterns and templates for new prompts;
using input from domain experts to use domain knowledge to suggest prompts spanning different healthcare scenarios and contexts;
performing A/B tests with candidate prompts with a large language model to assess response quality, clarity, specificity and adherence to healthcare compliance, to iteratively refine prompts based on test results.

4. The method of claim 1, wherein applying one or more machine learning models comprises performing in-memory analysis of transcript segments of the one or more transcripts.

5. The method of claim 1, wherein the one or more machine learning models are trained on healthcare terminology, medications and treatments to output healthcare domain-specific data.

6. The method of claim 1, wherein the plurality of digital service channels includes two or more channels selected from the group consisting of:
(i) a phone channel for interaction with agents trained to respond about benefits, claims and providers;
(ii) an email or secure messaging channel for written inquiries about benefits, claims, healthcare documents, including attachments for evidence of claim, explanation of benefits statements;
(iii) a chat or instant messaging channel for real-time interaction to obtain healthcare related information;
(iv) a web portal channel for secure online accounts to view benefits, check claim status, order identifier cards, updating contact information, uploading claims and documents and
(v) a social media channel for responding to public inquiries, providing updates during events impacting members.

7. The method of claim 1, wherein the one or more transcripts include:
(i) text related to healthcare topics including claims, benefits, insurance plans, coverage, medical terminology, and regulations;
(ii) at least some data with protected health information;
(iii) communication between patients, insurance companies and healthcare providers; and
(iv) speech and text data, including telephonic operations and call recordings.

8. The method of claim 1, wherein obtaining the one or more transcripts comprises interfacing with one or more third-party provider computers to receive text and/or speech data.

9. The method of claim 1, further comprising:
storing the one or more transcripts in a cloud object storage; and
cataloging and storing the metadata in a NoSQL database or persistent key-value datastore for replication, autoscaling, encryption at test, and on-demand backup.

10. The method of claim 9, wherein applying one or more machine learning models comprises performing in-memory analysis of transcript segments of the one or more transcripts, including, upon availability of the metadata, initiating a Kubeflow-based job, deploying a plurality of pods, each pod processing transcript segments, based on metadata from a document database and the one or more transcripts.

11. The method of claim 10, further comprising:
storing, by the plurality of pods, the channel-specific insights in a cloud object storage as encrypted files, using 256 AES encryption.

12. The method of claim 1, wherein obtaining the one or more transcripts comprises optimizing transcript processing using a read-optimized document database as an intermediary cache, wherein an hourly ETL job, orchestrated via Airflow, activates either a transient EMR cluster or an AWS Glue job, thereby identifying and migrating new records into a database.

13. The method of claim 1, wherein applying data filtering comprises:
filtering the channel-specific insights and the member-specific data by a plurality of parameters, including time, topic, and sentiment.

14. The method of claim 1, further comprising:
generating a data visualization based on the analytical insights, the data visualization subject to a predetermined latency.

15. The method of claim 1, further comprising:
indexing the analytical insights and/or the one or more transcripts chronologically for real-time querying.

16. The method of claim 1, further comprising:
providing one or more application programming interfaces (APIs) for (i) retrieving and/or (ii) interpreting user interface filters to query, the one or more call transcripts, the channel-specific insights, and/or the analytical insights.

17. The method of claim 1, wherein the one or more transcripts combines text or speech data obtained from the plurality of digital service channels, wherein at least two of the plurality of digital service channels generate text or speech in distinct format or structure.

18. The method of claim 1, wherein the one or more transcripts include text that is protected health information that is anonymized and is compliant with HIPAA and/or privacy regulations.

19. The method of claim 1, wherein generating the analytical insights comprises integrating interactions across multiple health service channels for a same member over time.

20. A computer system comprising:
one or more processors;
a display; and
memory;
wherein the memory stores one or more programs configured for execution by the one or more processors, and the one or more programs comprising instructions for:
obtaining one or more transcripts corresponding to a plurality of digital service channels;
generating one or more channel-specific prompts for the one or more transcripts based on each digital service channel corresponding to a respective transcript and metadata extracted from the one or more transcripts;
inputting the one or more channel-specific prompts to one or more machine learning models to obtain channel-specific insights; and
generating and displaying analytical insights by integrating the channel-specific insights with member-specific healthcare data, using sentiment analysis and data filtering.
